(19) United States
(12) Patent Application Publication
(10) Pub. No.: US 2008/0004343 A1
(43) Pub. Date: Jan. 3, 2008

(54) Stable polymorphs of (E)-N,N-diethyl-2-cyano-3-(3,4-dihydroxy-5-nitrophenyl)acrylamide

(75) Inventors: Siddiqui Mohammed Jaweed Mukarram, Aurangabad (IN); Rashid Abdul Rehman Khan, Aurangabad (IN); Ram Prasad Yadav, Aurangabad (IN); Zakir Gafoor Shaikh, Aurangabad (IN)

Correspondence Address: IPLM GROUP, P.A., POST OFFICE BOX 18455, MINNEAPOLIS, MN, 55418, US

(73) Assignee: Wockhardt Limited

(21) Appl. No.: 11/478,324

(22) Filed: Jun. 29, 2006

(51) Int. Cl. A61K 31/277

(52) U.S. Cl. 514/521; 558/408

(57) ABSTRACT

The present invention provides stable crystalline polymorphic Form C and Form D of (E)-Entacapone, (E)-N,N-diethyl-2-cyano-3-(3,4-dihydroxy-5-henyl)acrylamide and processes for their preparation. The polymorphic Form C and Form D of (E)-Entacapone are characterized by specific Infra Red (IR) and X-ray powder diffraction peak values.

### CROSS-REFERENCE TO RELATED APPLICATION

This application claims priority from international patent application Ser. No. PCT/IB2003/006200 filed Dec. 29, 2003, and published in English on Jul. 21, 2005 as International Publication No. WO 2005/066117 A1, which are incorporated herein by reference.

### FIELD OF THE INVENTION

The present invention relates to stable crystalline polymorphic Form C and Form D of Entacapone, (E)-N,N-iethyl-2-cyano-3-(3,4-dihydroxy-5-nitrophenyl)acrylamide and an efficient method for their preparation. Polymorphic Form C of Entacapone (E)-isomer is obtained by simple work-up procedure. Polymorphic Form D is obtained by converting Form A or Form C of Entacapone into Form D. Polymorphic form C and D of (E)-Entacapone are characterized by specific Infra Red (IR) and X-ray powder diffraction peak values. (E)-isomer of Entacapone is an excellent inhibitor of Catechol-O-methyl transferase (COMT) enzyme.

### BACKGROUND OF THE INVENTION

The chemical name for Entacapone is N,N-diethyl-2-cyano-3-(3,4-dihydroxy-5-nitrophenyl)acrylamide. U.S. patent application Ser. No. 4,963,590 and British Patent Application No. 2,200,109 A state that Entacapone is a potent inhibitor of catechol-O-methyl-transferase (COMT) enzyme. Entacapone has also been indicated for the treatment of Parkinsons disease. Catechol-O-methyltransferase (COMT) enzyme catalyzes the methyl group from S-adenosyl-L-methionine to a number of compounds with catechol structures. This enzyme is important in the extraneuronal inactivation of catecholamines and drugs with catechol structures. COMT is an important enzyme involved in the metabolism of catecholamines. The COMT enzyme is present in most tissues, both in the periphery and in the central nervous system. The highest activities are found in the liver, intestine and kidneys.

U.S. Pat. No. 4,963,590 (“the '590 patent”) discloses a process for the preparation of N,N-diethyl-2-cyano-3-(3,4-dihydroxy-5-nitrophenyl)acrylamide (Formula I). The synthetic process disclosed in the '590 patent includes the condensation of 3,4-dihydroxy-5-nitrobenzaldehyde (Formula II) and N,N-diethylcyano acetamide (Formula III) in anhydrous ethanol as shown below:

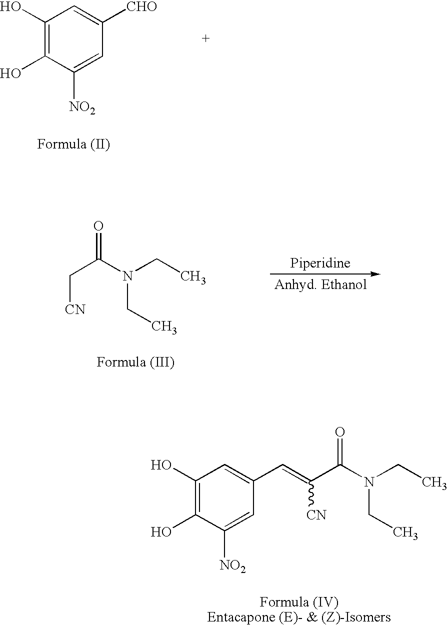

The catalyst used in the process described in the '590 patent synthesis was piperidine acetate. The Entacapone synthesized was obtained in 73% yield having a mixture of two geometrical isomeric forms, i.e., (E)- and (Z)-isomers. The separation of the isomers prepared is not disclosed in the '590 patent.

In addition, U.S. Pat. No. 5,135,950 (“the '950 patent”) discloses that (E)- and (Z)-isomers having the structural formula:

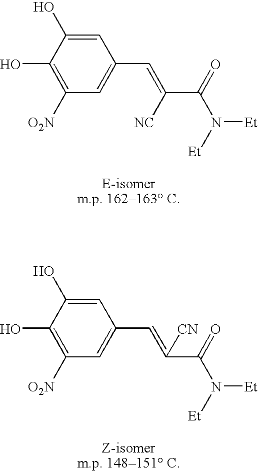

are obtained as mixture in the ratio of about 70-80% to about 30-20%, respectively. As revealed by X-Ray crystallography, (E)-N,N-diethyl-2-cyano-3-(3,4-dihydroxy-5-nitrophenyl)acrylamide may exist at least in two polymorphic forms A and B. The (Z)-isomer as well as polymorphic form B of the (E)-isomer has been shown to be unstable under the influence of heat or acids. Similarly, the polymorphic form B of the (E)-isomer isomerizes slowly to the polymorphic form A on standing at room temperature. Crude Entacapone synthesized from conventional solvents, such as hydrocarbons, alcohols or esters, e.g., benzene, toluene, methanol, ethanol, ethyl acetate, isopropyl acetate, etc., provide a very complicated mixture of different geometric isomers and/or polymorphic forms are generally obtained that interfere with the characterization and standardization of the drug substance. The bioavailability of the drug may be also influenced by polymorphism and geometrical isomerism.

The '950 patent discloses that “crystallographically essentially pure” and stable polymorphic Form A of (E)-N,N-diethyl-2-cyano-3-(3,4-dihydroxy-5-nitrophenyl)acrylamide may be obtained, when the crude product of Entacapone is re-crystallized from lower aliphatic carboxylic acids such as formic acid or acetic acid with a catalytic amount of hydrochloric hydrobromic acid as shown below:

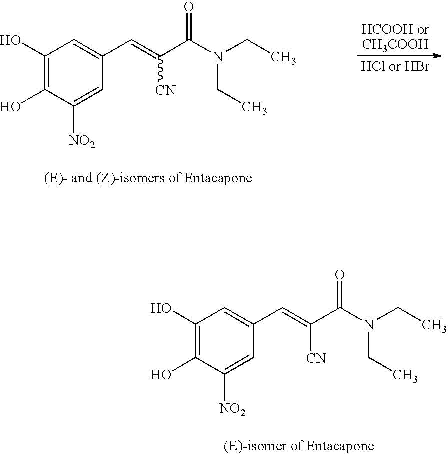

The '950 patent method states that this procedure allows the large scale production of homogeneous and crystallographically essentially pure polymorphic pure form A of (E)-N,N-diethyl-2-cyano-3-(3,4-dihydroxy-5-nitrophenyl)acrylamide. In the '950 patent, “crystallographically essentially pure” is stated to mean that the polymorphic form A of (E)-N,N-diethyl-2-cyano-3-(3,4-dihydroxy-5-nitrophenyl)acrylamide containing a maximum of 3% and preferably a maximum of 2% of other polymorphic forms or the Z-isomer.

Entacapone is used as a potent inhibitor of catechol-O-methyl transferase enzyme. The present invention provides a method for preparing new polymorphic Form C and Form D of (E)-N,N-diethyl-2-cyano-3-(3,4-dihydroxy-5-nitrophenyl)-acrylamide. Form C may be obtained in high purity without isolating crude solid of isomeric mixture of N,N-diethyl-2-cyano-3-(3,4-dihydroxy-5-nitrophenyl)acrylamide, directly by simple crystallization technique.

### SUMMARY OF THE INVENTION

The present invention is directed to an inhibitor compound of catechol-O-methyl-transferase having therapeutic value and a process for the manufacture thereof. In a first embodiment, the invention is directed to a crystallographically pure polymorphic Form C and Form D of Entacapone, (E)-N,N-diethyl-2-cyano-3-(3,4-dihydroxy-5-nitrophenyl)acrylamide having Formula I,

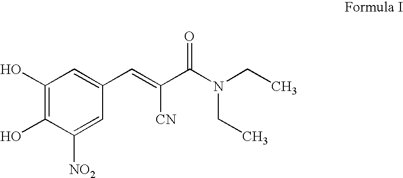

In another embodiment, the invention provides a process for the preparation of (E)-Entacapone Form C, comprising reacting 3,4-dihydroxy-5-nitrobenzaldehyde with N,N-diethylcyanoacetamide in alcohol and the presence of a base, followed by treatment with acetic acid and crystallization, from a mixture of alcohols.

In another embodiment, the invention is provides a process for the preparation of (E)-Entacapone Form D. The polymorphic Form D is prepared by converting (E)-N,N-diethyl-2-cyano-3-(3,4-dihydroxy-5-nitrophenyl)acrylamide polymorphic Form A or polymorphic Form C. The purity of polymorphic Form C and Form D have been assessed by HPLC and characterized by Infra Red Spectroscopy and X-Ray powder diffraction (XRD) techniques.

### BRIEF DESCRIPTION OF THE FIGURES

FIG. 1 illustrates the HPLC purity of the (E)-Entacapone of the present invention.

FIGS. 2 and 3 illustrate the Infra Red absorption bands for polymorphic Form C and Form D of the (E)-Entacapone of the present invention.

FIGS. 4 and 5 illustrate the X-ray powder diffraction pattern for polymorphic form C and D of the present invention.

### DETAILED DESCRIPTION OF THE INVENTION

The present invention provides an anti-catechol-O-methyl transferase compound in different polymorphic forms having therapeutic value and processes for their manufacture. In particular, the present invention is directed to the new polymorphic Form C and Form D of Entacapone, (E)-N,N-diethyl-2-cyano-3-(3,4-dihydroxy-5-nitrophenyl)acrylamide and processes for the preparation of such compound in different polymorphic forms.

The present invention provides a method for the manufacture of poly-morphic morphic Form C of (E)-N,N-diethyl-2-cyano-3-(3,4-dihydroxy-5-nitrophenyl)acrylamide. The product is obtained in highly pure form using absolute alcohol without isolating a crude solid. In another embodiment, the present invention provides a number of methods to prepare polymorphic Form D of (E)-Entacapone using different solvent combinations. The processes include dissolving “crystallographically pure Form A” or “crystallographically essentially pure Form A” or “Form C” in a lower carboxylic acid or highly polar water miscible organic solvent or mixtures thereof.

As used herein, the term “crystallographically essentially pure” means that the polymorphic form A of (E)-N,N-diethyl-2-cyano-3-(3,4-dihydroxy-5-nitrophenyl)acrylamide acrylamide contains a maximum of about 3% and preferably a maximum of about 2% of other polymorphic forms or of the Z-isomer.

In another embodiment, a process is provided for the manufacture of (E)-N,N-diethyl-2-cyano-3-(3,4-dihydroxy-5-nitrophenyl)acrylamide which includes the condensation of 3,4-Dihydroxy-5-nitrobenzaldehyde (Formula II) with N,N-diethylcyanoacetamide (Formula III) in presence of a base in alcoholic solution.

The synthetic method for preparing the crude Entacapone is shown below:

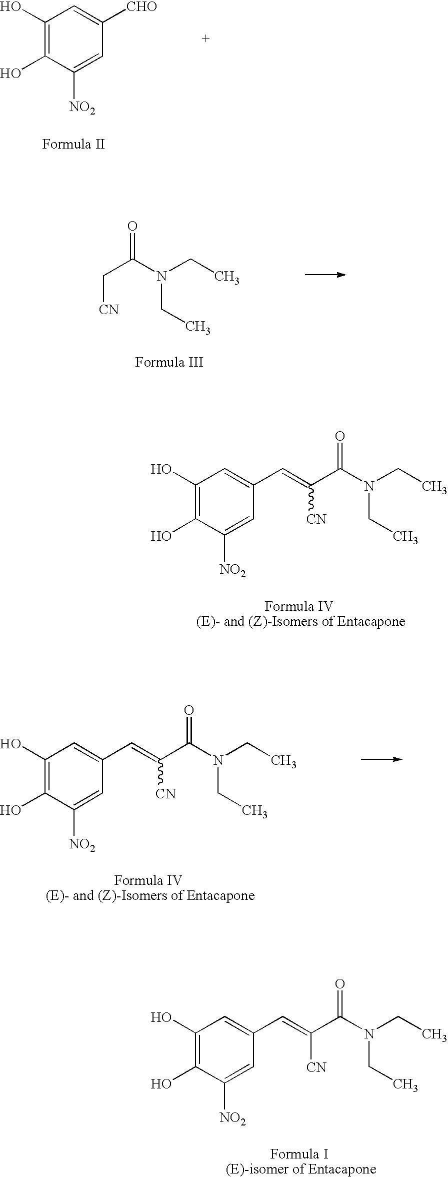

In another embodiment, the present invention, (E)-N,N-diethyl-2-cyano-3-(3,4-dihydroxy-5-nitrophenyl)acrylamide and its polymorphic Form C and Form D are prepared according the following synthetic reaction scheme:

### A. Reaction of 3,4-Dihydroxy-5-nitrobenzaldehyde and N-N-Diethylcyano-acetamide to form (E)-Entacapone Form C

3,4-Dihydroxy-5-nitrobenzaldehyde (Formula II) is reacted with N,N-diethylcyano-acetamide diethylcyano-acetamide (Formula III) in presence of a base in a solvent, to form a mixture of (E)- and (Z)-isomers of N,N-diethyl-2-cyano-3-(3,4-dihydroxy-5-nitrophenyl)acrylamide. Suitable solvents for this synthetic reaction include, but are not limited to, aliphatic alcohols, e.g., methanol, ethanol, isopropanol, isobutanol or n-butanol, more particularly isopropanol or ethanol. Suitable bases include, but are not limited to, amines, e.g., piperidine, N-methylmorpholine, morpholine, pyridine or piperazine, preferably piperidine. After the reaction is complete, aliphatic acid, preferably acetic acid, is added to the reaction mixture. Dilution of the reaction mixture with alcohol and cooling to from about 0° C. to 35° C. provides stable and pure polymorphic Form C of pure (E)-isomer (essentially without Z-isomer contamination) in 99.45% HPLC purity. In the present invention, the product, the mixture of isomers, is not isolated in its crude form; it is rather crystallized in reaction mixture itself containing ethyl alcohol.

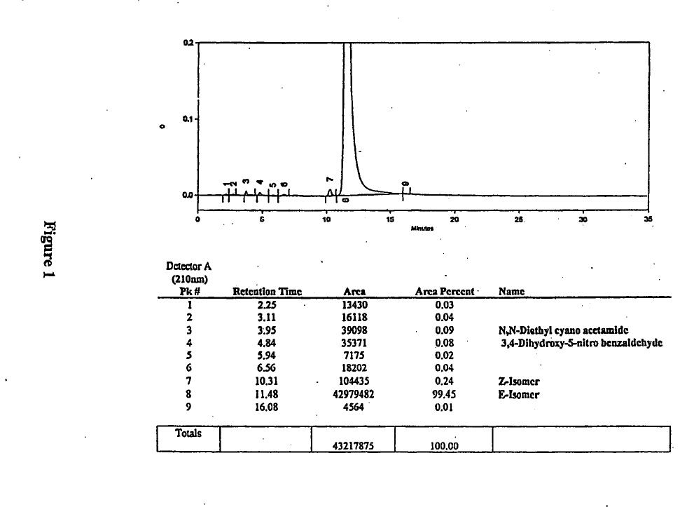

U.S. Pat. No. 5,135,950 states that crude mixture can not afford desired purity using lower aliphatic alcohols, esters or hydrocarbons, e.g. ethanol, 2-propanol, ethyl acetate, or toluene and a very complicated mixture of different geometrical isomers and/or polymorphic forms are generally obtained. Surprisingly, the present method provides pure product by very simple operation. HPLC purity of the product of present invention, i.e., (E)-Entacapone is more than 99% while (Z)-isomer is found in less than 0.3% (FIG. 1).

The specification of HPLC profile of the present invention is mentioned below:

Column: HICHROM HIRPB (250×4.6 m, 5μ)

Wavelength: 210 nm

Flow rate: 1.0 ml /minute

Temperature: Ambient

Buffer: 1 ml H₃PO₄ in 1000 ml distilled water

Mobile phase: Buffer (65%): Acetonitrile (35%)

Sample preparation: 0.5 mg/ml in mobile phase

Retention Time: (E)-Isomer=11.5 minutes,Z-Isomer=10.3 minutes

### B. Preparation of Polymorohic Form D of (E)-N,N-Diethyl-2-cyano-3-(3,4-dihydroxy-5-nitrophenyl)acrylamide

Crystallographically pure Form A of (E)-N,N-diethyl-2-cyano-3-(3,4-dihydroxy-5-nitrophenyl)acrylamide is dissolved in a polar organic solvent. Suitable polar solvents include aliphatic organic amides, preferably N,N-dimethylformamide or N,N-Dimethyl acetamide. Entacapone solution is added slowly and carefully to vigorously stirred, chilled water. The reaction mixture is stirred additionally for about 1 to about 5 hours. The polymorphic Form D Entacapone formed as crystals is washed with water and isopropyl ether. The product obtained is dried under vacuum to provide the title product in 90% yield.

In another embodiment, crystallographically pure Form C of (E)-N,N-diethyl-2-cyano-3-(3,4-dihydroxy-5-nitrophenyl)acrylamide is dissolved in aliphatic alcohol, preferably in ethanol, to obtain a clear solution. The alcoholic solution is added to vigorously stirred, chilled water and stirred additionally for about 1 to about 5 hours. The title compound, Form C of (E)-N,N-diethyl-2-cyano-3-(3,4-dihydroxy-5-nitrophenyl)acrylamide, is isolated by filtration, washing with water and isopropyl ether, followed by vacuum drying. The (E)-Entacapone Form D is obtained as a crystalline material in 88% yield.

In another embodiment, crystallographically essentially pure Form A of (E)-N,N-diethyl-2-cyano-3-(3,4-dihydroxy-5-nitrophenyl)acrylamide, is dissolved in a sulfoxide, preferably in dimethylsulfoxide. The solution is added to vigorously stirred, chilled water and stirred additionally for about 1 to about 5 hours. The title compound, Form D of (E)-N,N-diethyl-2-cyano-3-(3,4-dihydroxy-5-nitrophenyl)acrylamide, is isolated by filtration, washing with water and isopropyl ether, followed by vacuum drying. The (E)-Entacapone Form D is obtained as a crystalline material in about 91% yield and in greater than 99% HPLC purity.

### C. Characterization of (E)-N,N-Diethyl-2-cyano-3-(3,4-dihydroxy-5-nitrophenyl)acrylamide Form C and D

Crystalline Form C and D of (E)-N,N-diethyl-2-cyano-3-(3,4-dihydroxy-5-nitrophenyl)acrylamide was characterized by Infra Red (IR) Spectrum, X-Ray powder diffraction (XRD) and melting points, and the purity is tested HPLC. For comparison purposes, certain of these analyses have was been performed for the corresponding polymorphic form A of Entacapone.

### Infra Red Spectrum

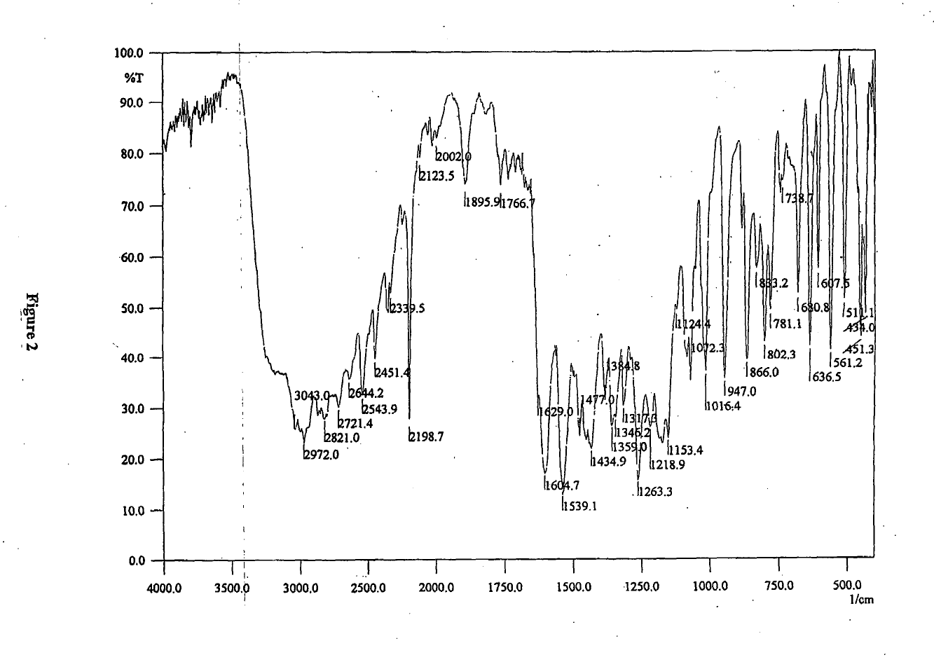

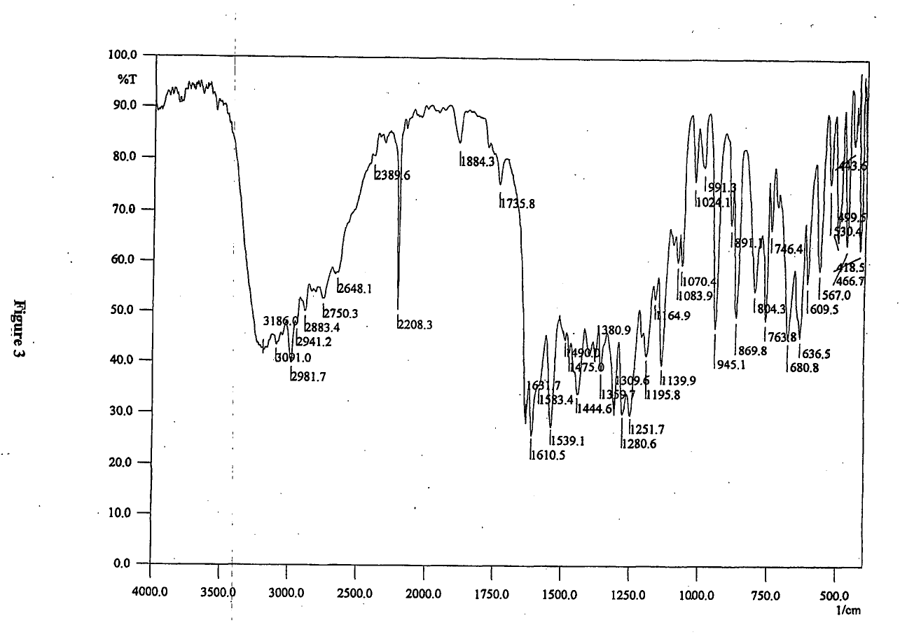

FIGS. 2 and 3 illustrate the Infra Red absorption bands for polymorphic Form C and Form D of (E)-N,N-diethyl-2-cyano-3-(3,4-dihydroxy-5-nitrophenyl)-acrylamide, respectively. The IR Peaks for Entacapone Form A are taken from the '590 patent and included for comparison purposes. A comparison of the IR peak values of the polymorphic forms of (E)-Entacapone is set forth in Table 1.

TABLE 1
Infra Red Peaks (cm⁻¹) for the Polymorphic Forms A, C and D of
(E)-N,N-Diethyl-2-cyano-3-(3,4-dihydroxy-5-nitrophenyl)acrylamide

| Sample No. | Form A | Form C | Form D |
|---|---|---|---|
| 1 | 3339 | 3043 | 3186 |
| 2 | 3092 | 2972 | 3091 |
| 3 | 3066 | 2821 | 2982 |
| 4 | 3039 | 2721 | 2941 |
| 5 | 2981 | 2644 | 2883 |
| 6 | 2938 | 2544 | 2750 |
| 7 | 2217 | 2451 | 2648 |
| 8 | 1628 | 2340 | 2390 |
| 9 | 1607 | 2199 | 2208 |
| 10 | 1580 | 2124 | 1884 |
| 11 | 1544 | 2002 | 1736 |
| 12 | 1512 | 1896 | 1632 |
| 13 | 1441 | 1767 | 1611 |
| 14 | 1377 | 1629 | 1583 |
| 15 | 1298 | 1605 | 1539 |
| 16 | 1281 | 1539 | 1490 |
| 17 | 1210 | 1477 | 1475 |
| 18 | 1165 | 1435 | 1445 |
| 19 | 1150 | 1385 | 1381 |
| 20 | 800 | 1359 | 1360 |
| 21 | 779 | 1346 | 1310 |
| 22 | 740 | 1317 | 1281 |
| 23 | | 1263 | 1252 |
| 24 | | 1219 | 1196 |
| 25 | | 1153 | 1140 |
| 26 | | 1124 | 1165 |
| 27 | | 1072 | 1084 |
| 28 | | 1016 | 1070 |
| 29 | | 947 | 1024 |
| 30 | | 866 | 991 |
| 31 | | 833 | 945 |
| 32 | | 802 | 891 |
| 33 | | 781 | 870 |
| 34 | | 739 | 804 |
| 35 | | 681 | 764 |
| 36 | | 637 | 746 |
| 37 | | 608 | 681 |
| 38 | | 561 | 637 |
| 39 | | 511 | 610 |
| 40 | | | 567 |
| | | | 530 |

### X-Ray Powder Diffraction

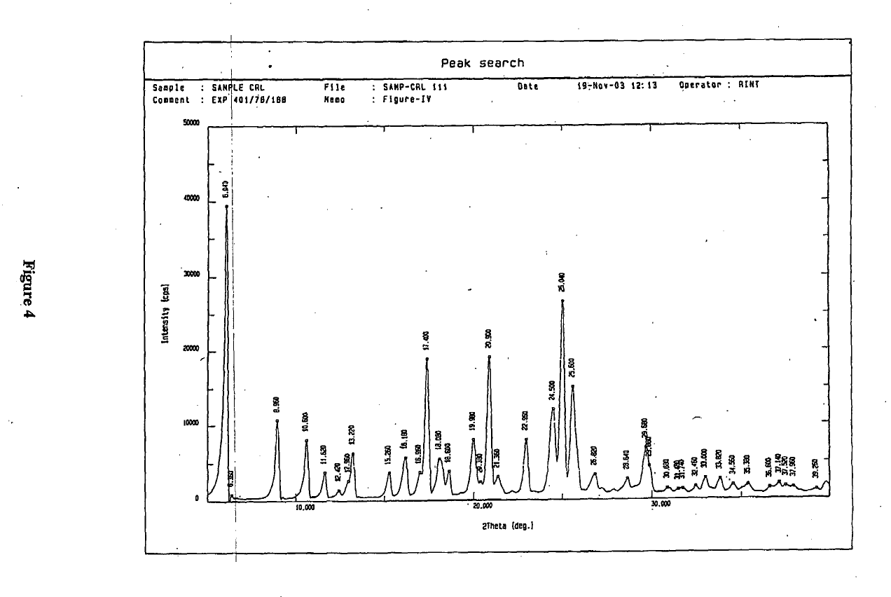

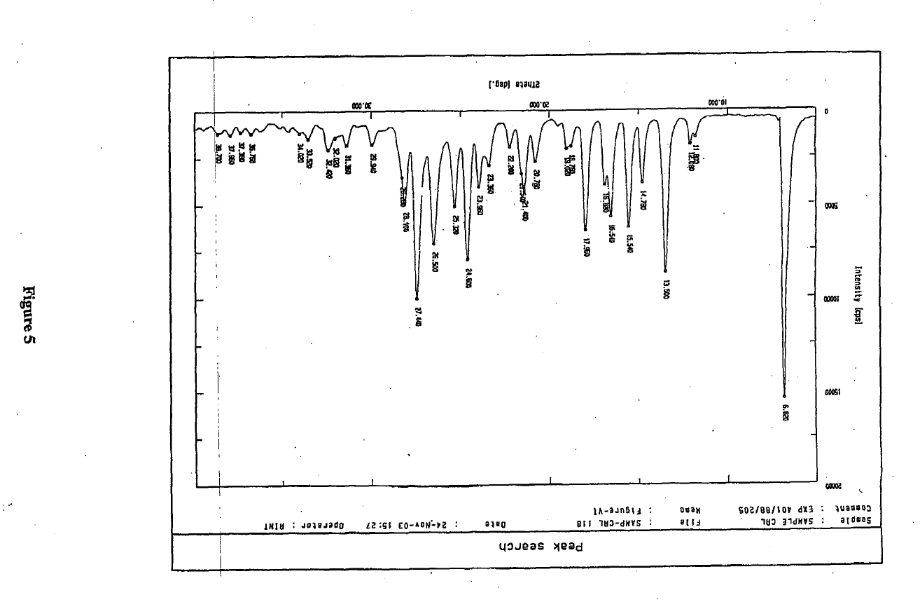

FIGS. 4 and 5 illustrate the X-ray powder diffraction pattern for polymorphic form C and D of (E)-N,N-diethyl-2-cyano-3-(3,4-dihydroxy-5-nitrophenyl)acrylamide, respectively. A comparison of the diffraction peaks, designated by “2θ” and expressed in degrees, is set forth in Table 2.

TABLE 2
XRD Peaks for the Polymorphic Form A, C and D of (E)-N,N-Diethyl-
2-cyano-3-(3,4-dihydroxy-5-nitrophenyl)acrylamide

| Sample No. | Form A | Form C | Form D |
|---|---|---|---|
| 1 | 3.680 | 6.040 | 6.820 |
| 2 | 9.040 | 6.360 | 11.800 |
| 3 | 11.840 | 8.960 | 12.080 |
| 4 | 13.541 | 10.600 | 13.500 |
| 5 | 14.060 | 11.620 | 14.780 |
| 6 | 15.820 | 12.420 | 15.540 |
| 7 | 16.320 | 12.960 | 16.540 |
| 8 | 18.220 | 13.220 | 16.880 |
| 9 | 18.459 | 15.260 | 17.960 |
| 10 | 18.720 | 16.180 | 18.760 |
| 11 | 18.940 | 16.960 | 19.020 |
| 12 | 20.041 | 17.400 | 20.760 |
| 13 | 20.380 | 18.080 | 21.400 |
| 14 | 21.140 | 18.600 | 21.540 |
| 15 | 21.939 | 19.980 | 22.200 |
| 16 | 22.901 | 20.380 | 23.360 |
| 17 | 23.340 | 20.900 | 23.960 |
| 18 | 23.960 | 21.360 | 24.600 |
| 19 | 24.480 | 22.960 | 25.320 |
| 20 | 26.343 | 24.500 | 26.500 |
| 21 | | 25.040 | 27.440 |
| 22 | | 25.600 | 28.100 |
| 23 | | 26.820 | 28.260 |
| 24 | | 28.640 | 29.940 |
| 25 | | 29.680 | 31.360 |
| 26 | | 29.880 | 32.020 |
| 27 | | 30.880 | 32.420 |
| 28 | | 31.480 | 33.520 |
| 29 | | 31.740 | 34.020 |
| 30 | | 32.460 | 36.760 |
| 31 | | 33.000 | 37.380 |
| 32 | | 33.820 | 37.960 |
| 33 | | 34.560 | 38.700 |
| 34 | | 35.380 | |
| 35 | | 36.600 | |
| 36 | | 37.140 | |
| 37 | | 37.520 | |
| 38 | | 37.960 | |
| 39 | | 39.260 | |

### EXAMPLES

The following examples illustrate the invention, but are not limiting thereof.

### Example 1

### Crystallographically pure (E)-N,N-Diethyl-2-cyano-3-(3,4-dihydroxy-5-nitrophenyl)acrylamide Form C

3,4-Dihydroxy-5-nitrobenzaldehyde (10.0 gm, 54.6 mmol), N,N-diethyl-cyanoacetamide (15.5 gm, 11.06 mmol) and piperidine (15.0 gm, 170.3 mmol) is charged to isopropanol (200 ml). The reaction mixture is heated at reflux for 12 to 15 hours until the starting material is disappeared. After the reaction is complete, the reaction mixture is cooled to room temperature and acetic acid glacial (15 ml) is added. The reaction mixture is concentrated, followed by dilution with ethyl alcohol (50 ml). The mixture is stirred overnight and filtered. The isolated product is dried under vacuum to provide crystallographically pure Form C of crystalline (E)-N, N-diethyl-2-cyano-3-(3,4-dihydroxy-5-nitrophenyl)acrylamide with melting point of 156-160° C. The title product is obtained as a crystalline solid in 75.5% yield. HPLC Purity=99.45% (Z-isomer 0.24%). Mass Spectra=m+1 306.1 (100%).

### Example 2

### Crystallographically pure (E)-N,N-Diethyl-2-cyano-3-(3,4-dihydroxy-5-nitrophenyl)acrylamide Form D

Crystallographically pure form A of (E)-N,N-diethyl-2-cyano-3-(3,4-dihydroxy-5-nitrophenyl)acrylamide (5.0 gm) is dissolved in N,N-dimethylformamide (25 ml) to provide a clear solution. The solution is slowly and carefully added to vigorously stirred, chilled water (200 ml). Stirring is continued for an additional two hours to obtain a precipitate. Precipitated product is isolated by filtration, followed by washing with water and isopropyl ether. The title product is dried under vacuum to obtain, (E)-N,N-diethyl-2-cyano-3-(3,4-dihydroxy-5-nitrophenyl)acrylamide Form D, in crystalline form, in 90% yield having melting point 158-162° C., and a 90.0% yield.

### Example 3

### Crystallographically pure (E)-N,N-Diethyl-2-cyano-3-(3,4-dihydroxy-5-nitrophenyl)acrylamide Form D

Crystallographically pure Form C of (E)-N,N-diethyl-2-cyano-3-(3,4-dihydroxy-5-nitrophenyl)acylamide (5.0 gm) is dissolved in ethanol (45 ml). The clear solution is added to vigorously stirred, chilled water (200 ml) and stirring is continued for an additional two hours. The precipitated product is isolated by filtration, followed by washing with water and isopropyl ether. The (E)-Entacapone Form D obtained is dried under vacuum. The title product is obtained in crystalline form in 88.0% yield.

### Example 4

### Crystallographically pure (E)-N,N-Diethyl-2-cyano-3-(3,4-dihydroxy-5-nitrophenyl)-acrylamide Form D

“Crystallographically essentially pure form A” of (E)-N,N-diethyl-2-cyano-3-(3,4-dihydroxy-5-nitrophenyl)acrylamide (5.0 gm) is dissolved in N,N-dimethyl acetamide (25 ml). The clear solution is added slowly and carefully to vigorously stirred, chilled water (200 ml). Stirring is continued for a further two hours. Precipitated product is isolated by filtration followed by washing with water and isopropyl ether. The product obtained is dried under vacuum. The title compound is obtained in crystalline form in 91.0% yield.

### Example 5

### Crystallographically pure (E)-N,N-Diethyl-2-cyano-3-(3,4-dihydroxy-5-nitrophenyl)-acrylamide Form D

“Crystallographically essentially pure form A” of (E)-N,N-diethyl-2-cyano-3-(3,4-dihydroxy-5-nitrophenyl)acrylamide (5.0 gm) is dissolved in dimethyl-sulphoxide (25 ml) to make solution. Clear solution is added to vigorously stirred, chilled water (200 ml). Stirring is continued for further two hours and precipitated product is isolated by filtration followed by washing with water and isopropyl ether. The (E)-Entacapone obtained by this method is dried under vacuum. The title compound is obtained in crystalline form in 90% yield.

All references cited herein are expressly incorporated herein by reference in their entirety into this disclosure. Illustrative embodiments of this disclosure are discussed and reference has been made to possible variations within the scope of this disclosure. These and other variations and modifications in the disclosure will be apparent to those skilled in the art without departing from the scope of the disclosure, and it should be understood that this disclosure and the claims shown below are not limited to the illustrative embodiments set forth herein.

1-40. (canceled)

41. Crystalline Form D of entacapone; or a pharmaceutically acceptable salt thereof.

42. The crystalline Form D of entacapone of claim 41, having characteristic X-ray diffraction peaks at 2-theta values of about 6.820, 14.780, 15.540, 16.540, 16.880, 17.960, 20.760, 21.400, 21.540 and 22.200.

43. The crystalline Form D of entacapone of claim 42, further characterized by X-ray diffraction peaks at 2-theta values of about 11.800, 12.080, 13.500, 18.760, 19.020, 23.360, 23.960, 24.600, 25.320, 26.500, 27.440, 28.100, 28.260, 29.940, 31.360, 32.020, 32.420, 33.520, 34.020, 36.760, 37.380, 37.960 and 38.700.

44. The crystalline Form D of entacapone of claim 41, wherein the entacapone has the infrared spectrum of FIG. 3.

45. The crystalline Form D of entacapone of claim 44, having characteristic infrared peaks at about 3186, 3091, 2982, 2941, 2883, 2750, 2648, 2390, 2208, 1884, 1736, 1632, 1611, 1583, 1539, 1490, 1475, 1445, 1381, 1360, 1310, 1281, 1252, 1196, 1140, 1165, 1084, 1070, 1024, 991, 945, 891, 870, 804, 764, 746, 681, 637, 610, 567 and 530 cm-1.

46. A process for the preparation of crystalline Form D of entacapone comprising:
a) obtaining a solution of entacapone in a suitable organic solvent;
b) contacting the solution of step a) with water; and
c) isolating the crystalline Form D of entacapone.

47. The process of claim 46, wherein the organic solvent is a polar solvent.

48. The process of claim 47, wherein the polar solvent comprises one or more of ethanol, N,N-dimethylformamide, N,N-dimethylacetamide, and dimethyl sulphoxide.

49. The process of claim 46, wherein the water is pre-cooled to less than 10 deg C.

50. The process of claim 46, wherein the crystalline Form D of entacapone has purity greater than 99.4% by HPLC.

51. A pharmaceutical composition comprising a therapeutically effective amount of crystalline Form D of entacapone and a pharmaceutically acceptable carrier.

52. A method of inhibiting catechol-O-methyl-transferase enzyme in a warm blooded animal, the method comprising administering a pharmaceutical composition comprising crystalline Form D of entacapone and a pharmaceutically acceptable carrier.
